(19) United States
(12) Patent Application Publication
(10) Pub. No.: US 2006/0241344 A1
(43) Pub. Date: Oct. 26, 2006

(54) Intra-abdominal surgical method and associated apparatus

(75) Inventor: Peter J. Wilk, New York, NY (US)

Correspondence Address: R. Neil Sudol, 714 Colorado Avenue, Bridgeport, CT, 06605-1601, US

(73) Assignee: WILK PATENT, LLC, New York, NY (US)

(21) Appl. No.: 11/385,352

(22) Filed: Mar. 21, 2006

Related U.S. Application Data
(60) Provisional application No. 60/670,513, filed on Apr. 12, 2005.

(51) Int. Cl. A61B 1/00

(52) U.S. Cl. 600/114

(57) ABSTRACT

A distal end portion of an endoscope insertion member is inserted into a hollow internal organ of a patient, the endoscope insertion member being at least partially encased by a sheath provided with at least one longitudinally extending tubular channel. An incision or perforation is formed in a wall of the organ. The distal end portion of the endoscope insertion member is moved through the incision or perforation into a hollow internal space inside the patient. Thereafter an insufflation gas is delivered through the channel of the endoscope sheath into the internal space.

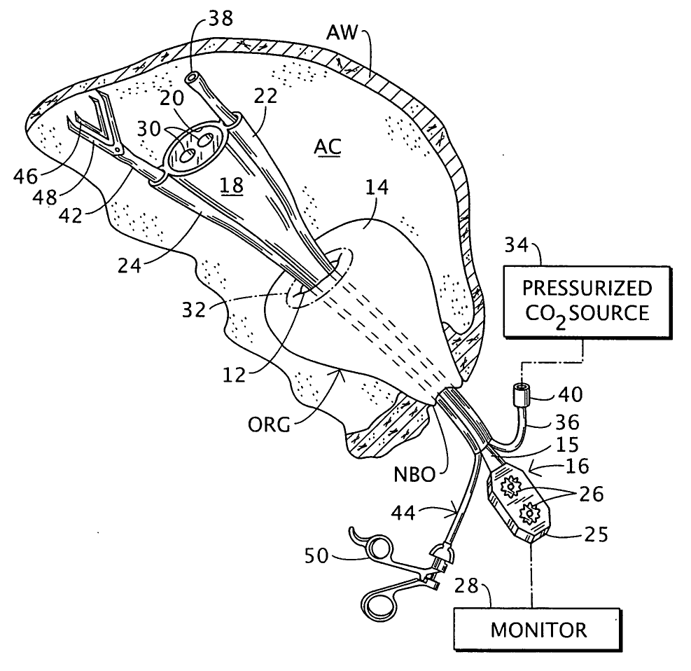

### CROSS-REFERENCE TO RELATED APPLICATION

This application claims the benefit of U.S. Provisional Patent Application No. 60/670,513 filed Apr. 12, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 may be termed “trans-organ operations” where access to an internal body cavity such as the abdominal cavity is attained via an internal organ such as the stomach, colon, or vagina. The abdominal cavity may be insufflated prior to the insertion of the distal end of the endoscope into the abdominal cavity. Pneumoperitoneum may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

One object of trans-organ surgery is to reduce trauma to the individual. One means for accomplishing this end result is to reduce the number of incisions that must be made in the patient to obtain access to a surgical site in the abdominal cavity. Hospital convalescence stays are even shorter.

### OBJECTS OF THE INVENTION

It is an object of the present invention to provide improvements on the afore-described surgical procedures.

It is another object of the present invention to provide a method and/or associated apparatus for maintaining pneumoperitoneum.

It is a further object of the invention to provide a trans-organ surgical method that reduces the number of incisions made to obtain access to the abdominal cavity.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical method comprises, in accordance with the present invention, inserting a distal end portion of an endoscope insertion member into a hollow internal organ of a patient, where the endoscope insertion member is at least partially encased by a sheath provided with at least one longitudinally extending tubular channel. The method further comprises forming an incision or perforation in a wall of the organ, moving the distal end portion of the endoscope insertion member through the incision or perforation into a hollow internal space inside the patient, and thereafter delivering an insufflation gas through the channel of the endoscope sheath into the internal space.

This method eliminates the necessity for making a separate incision for forming and maintaining pneumoperitoneum. An endoscope may be inserted into the abdominal cavity through an incision made in the wall of an internal body cavity or organ by an insufflation needle. Pneumoperitoneum may be subsequently maintained via the same incision or artificial opening through which the endoscope extends.

The sheath may be provided with a plurality of longitudinally extending tubular channels, the method further comprising passing a distal end portion of an elongate surgical instrument through another of the channels into the internal space after the moving of the distal end portion of the endoscope into the internal space. The method may further comprise manipulating the surgical instrument from outside the patient to perform an operation on organic tissues in the internal space and using the endoscope to visually monitor the internal space during the manipulating of the surgical instrument. The sheath may be open at both the distal end and the proximal end. In that case, the using of the endoscope includes transmitting visible radiation through the distal opening.

The inserting of the distal end portion of the endsocope insertion member into the hollow internal organ may occur prior to the forming of the incision or perforation in the wall of the organ. In that case, the forming of the incision or perforation may include operating an incising instrument from outside the patient and using the endoscope to visually monitor the wall of the hollow internal organ during the operating of the incising instrument. Where the channel is one of a plurality of longitudinally extending tubular channels with which the sheath is provided, a distal end portion of the incising instrument is inserted into the hollow internal organ via one of the channels.

In one embodiment of the invention, the delivering of the insufflation gas includes inserting a tube through the channel and connecting the tube to a source of the insufflation gas.

Where the hollow internal organ communicates with the ambient environment via a natural body opening, the distal end portion of the endoscope insertion member is inserted into the hollow internal organ via the natural body opening. The hollow internal organ may be the stomach, the colon, the vagina, the urinary bladder, or a blood vessel.

A surgical kit utilizable in a minimally invasive surgical procedure with an endoscope insertion member having a distal end portion comprises, in accordance wit the present invention, a sheath, an incising instrument, and a coupling element. The sheath is provided with at least one longitudinally extending tubular channel and is disposable about the endoscope insertion member. The incising instrument has an elongate shaft and an operative tip at a distal end of the shaft for forming an incision or perforation in a wall of an internal organ of a patient. The instrument is insertable though a working channel extendable along the distal end portion of the endoscope insertion member, the working channel being a biopsy channel of the endoscope insertion member or the tubular channel in the sheath. The coupling element is connectable to the endoscope insertion member or the sheath at a proximal end thereof and also operatively connectable to a source of pressurized gas, for enabling delivery an insufflation gas along the endoscope into an internal space of a patient.

The tubular channel and the working channel may be among a plurality of working channels, including a plurality of longitudinally extending tubular channels provided along the sheath. The surgical kit further comprises an elongate surgical instrument insertable through one of the working channels, where the elongate surgical instrument has an operative tip different in structure and function from the operative tip of the incising instrument.

The sheath may be provided with an opening at a distal end enabling a transmission of visible radiation from the distal end portion of the endoscope insertion member in a distal direction away from the distal end portion of the endoscope insertion member.

Pursuant to a feature of the present invention, the coupling element is connected to a tube insertable through at least one of the tubular channel and the working channel.

A surgical kit comprises, in accordance with a further aspect of the present invention, a sheath for an endoscope, the sheath being provided with at least one longitudinally extending channel, and means for operatively connecting one end of the channel to a source of pressurized gas.

A surgical kit comprises, in accordance with another aspect of the present invention, a sheath for an endoscope, the sheath being provided with at least one longitudinally extending channel, and a tubular conduit insertable through the channel, the conduit having means at one end connectable to a source of pressurized gas. The kit may additionally comprise at least one elongate instrument having a shaft insertable through the channel.

### BRIEF DESCRIPTION OF THE DRAWING

The sole figure of the drawing is a schematic partial cross-sectional view of a person, showing an instrument assembly pursuant to the present invention, inserted into the patient for performing surgery in the abdominal cavity, in accordance with the present invention. Illustrated organs have been greatly simplified as anatomical details are irrelevant to explicating the invention.

### DETAILED DESCRIPTION

As shown in the drawing, an incision 12 is formed in a wall 14 of a hollow internal body organ ORG that communicates with the ambient space via a natural body opening NBO. Organ ORG may be the colon, the vagina, or the urinary bladder, while natural body opening NBO is the anus, the vaginal orifice, or the urethral opening.

Incision 12 is typically formed by first inserting a distal end portion of an insertion member 15 of an endoscope 16 into organ ORG via opening NBO. Endoscope 16 is provided with a sheath 18 having an opening 20 at a distal end and a plurality of longitudinally extending channels 22 and 24. Endoscope 16 includes a handpiece 25 at a proximal end, which carries a pair of turning knobs 26. Endoscope 16 is connected to a video monitor 28 for displaying an image of internal tissues obtained via optical elements 30 of the endoscope. The using of endoscope 16 includes transmitting visible radiation through optical elements 30 and distal sheath opening 20.

Upon inserting the distal end portion of endoscope insertion member 15 into organ ORG via natural body opening NBO, an incising instrument (not shown) is inserted through a channel 24 of sheath 18. The incising instrument is manipulated from outside the patient to form incision 12 in organ wall 14. Preferably, a port element 32 is installed in organ wall 14 to facilitate the insertion and removal of instrumentation through incision 12. Port 32 may take any suitable form. U.S. Pat. Nos. 5,297,536 and 5,458,131 describe at least one possible form.

After the deployment of port element 32, the distal end portion of endoscope insertion member 15 is moved through the port element and through incision or perforation 12 into a hollow internal space AC, such as the abdominal cavity, inside the patient. Thereafter an insufflation gas such as carbon dioxide is delivered from a pressurized gas source 34 through a tube or conduit 36 inserted at least partially into channel 22 of endoscope sheath 18. A distal end 38 of tube or conduit 36 may protrude from a distal end (not separately labeled) of sheath channel 22. A proximal end of tube or conduit 36 includes a connector 40 for coupling the tube to pressurized gas source 34. The insufflation gas delivered via tube or conduit 36 is sufficient to maintain pneumoperitoneum in the abdominal cavity AC, i.e., sufficient to maintain an abdominal wall AW of the patient sufficiently distended to enable the performance of a surgical procedure inside the abdominal cavity under observation via endoscope 16 and monitor 28.

Pneumoperitoneum may be generated initially through use of a Veress needle inserted through abdominal wall AW or through organ wall 14. In the latter procedure, incision 12 may be formed at the same location in wall 14 penetrated by the Veress needle. Endoscope insertion member 15 may be inserted into organ ORG prior to the use of the Veress needle to facilitate visual monitoring of the initial insufflation operation.

After the passage of the distal end portion of endoscope insertion member 15 (together with distal end portions of sheath 18 and channels or tubes 22 and 24), and an elongate shaft 42 of a surgical instrument 44 such as a stapler (with a staple 46) or a forceps may be inserted through channel 24 so that an operative tip 48 of instrument 44 is located inside abdominal cavity AC, as depicted in the drawing. A handle 50 of instrument 44 is then manipulated from outside the patient to perform a surgical operation inside abdominal cavity AC, while visualization of the operative tip 48 and a surgical site is attained via endoscope 16 and monitor 28.

Sheath 18 may be marketed in a kit including tube or conduit 36 and/or one or more surgical instruments 44. The instruments 44 of the kit may vary in accordance with different kinds of procedures that may be performed via trans-organ access as described above. Thus, a tubal ligation would require a clip applier. A bladder removal procedure could utilize a cutting and cauterization tool, a graspers and a tissue removal instrument (none shown in particular). Sheath 18 may include more than two channels 22 and 24 should more than one instrument be required simultaneously during a contemplated procedure.

Although the invention has been described in terms of particular embodiments and applications, one of ordinary skill in the art, in light of this teaching, can generate additional embodiments and modifications without departing from the spirit of or exceeding the scope of the claimed invention. For example, pneumoperitoneum may be maintained via an endoscope sheath where the associated endoscope is inserted through the abdominal wall, e.g., via a trocar sleeve or cannula. In addition, the access to the abdominal cavity AC may alternatively be had via the vascular system. In that case, an incision may be formed in the skin to access the vascular system. Furthermore, tube or conduit 36 may be omitted, provided that channel or tube 22 has a connector element for coupling.

Accordingly, it is to be understood that the drawings and descriptions herein are proffered by way of example to facilitate comprehension of the invention and should not be construed to limit the scope thereof.

What is claimed is:

1. A surgical method comprising:
inserting a distal end portion of an endoscope insertion member into a hollow internal organ of a patient, said endoscope insertion member being at least partially encased by a sheath provided with at least one longitudinally extending tubular channel;
forming an incision or perforation in a wall of said organ;
moving said distal end portion of said endoscope insertion member through said incision or perforation into a hollow internal space inside the patient; and
after the moving of said distal end portion into said internal space, delivering an insufflation gas through said channel into said internal space.

2. The method defined in claim 1 wherein said channel is one of a plurality of longitudinally extending tubular channels with which said sheath is provided, further comprising passing a distal end portion of an elongate surgical instrument through another of said channels into said internal space after the moving of said distal end portion into said internal space.

3. The method defined in claim 2, further comprising manipulating said surgical instrument from outside the patient to perform an operation on organic tissues in said internal space and using said endoscope to visually monitor said internal space during the manipulating of said surgical instrument.

4. The method defined in claim 3 wherein said sheath has an opening at a distal end, the using of said endoscope including transmitting visible radiation through said opening.

5. The method defined in claim 1 wherein the inserting of said distal end portion of said endsocope insertion member into said hollow internal organ occurs prior to the forming of said incision or perforation, the forming of said incision or perforation including operating an incising instrument from outside the patient and using said endoscope to visually monitor said wall of said hollow internal organ during the operating of said incising instrument.

6. The method defined in claim 5 wherein said channel is one of a plurality of longitudinally extending tubular channels with which said sheath is provided, a distal end portion of said incising instrument being inserted into said hollow internal organ via one of said channels.

7. The method defined in claim 1 wherein the delivering of said insufflation gas includes inserting a tube through said channel and connecting said tube to a source of said insufflation gas.

8. The method defined in claim 1 wherein said hollow internal organ communicates with the ambient environment via a natural body opening, said distal end portion of said endoscope insertion member being inserted into said hollow internal organ via said natural body opening.

9. The method defined in claim 1 wherein said hollow internal organ is taken from the group consisting of the stomach, the colon, the vagina, the urinary bladder, and a blood vessel.

10. A surgical kit comprising:
a sheath for an endoscope, said sheath being provided with at least one longitudinally extending channel; and
a tubular conduit insertable through said channel, said conduit having means at one end connectable to a source of pressurized gas.

11. The kit defined in claim 10, further comprising at least one elongate instrument having a shaft insertable through said channel.

12. The kit defined in claim 10 wherein said sheath defines a main lumen for receiving an endoscope insertion member, said lumen being open at each of two opposite ends.

13. The kit defined in claim 10 wherein said channel is one of a plurality of longitudinally extending channels included in said sheath.

14. A surgical kit comprising:
a sheath for an endoscope, said sheath being provided with at least one longitudinally extending channel; and
means for operatively connecting one end of said channel to a source of pressurized gas.

15. The kit defined in claim 14, further comprising at least one elongate instrument having a shaft insertable through said channel

16. The kit defined in claim 15 wherein said channel is one of a plurality of longitudinally extending channels included in said sheath, said shaft of said elongate instrument being insertable through the other of said longitudinally extending channels.

17. The kit defined in claim 14 wherein said sheath defines a main lumen for receiving an endoscope insertion member, said lumen being open at each of two opposite ends.

18. A surgical kit utilizable in a minimally invasive surgical procedure with an endoscope insertion member having a distal end portion, said kit comprising:
a sheath provided with at least one longitudinally extending tubular channel, said sheath being disposable about the endoscope insertion member;
an incising instrument having an elongate shaft and an operative tip at a distal end of the shaft for forming an incision or perforation in a wall of an internal organ of a patient, said instrument being insertable though a working channel extendable along the distal end portion of the endoscope insertion member, said working channel being taken from the group consisting of a biopsy channel of said endoscope insertion member and said tubular channel;
a coupling element connectable to said endoscope insertion member or said sheath at a proximal end thereof and also operatively connectable to a source of pressurized gas, for enabling delivery an insufflation gas along said endoscope into an internal space of a patient.

19. The surgical kit defined in claim 18 wherein said tubular channel and said working channel are among a plurality of working channels, said working channels including a plurality of longitudinally extending tubular channels provided along said sheath, further comprising an elongate surgical instrument through one of said working channels, said elongate surgical instrument having an operative tip different in structure and function from the operative tip of said incising instrument.

20. The surgical kit defined in claim 18 wherein said sheath has an opening at a distal end enabling a transmission of visible radiation from the distal end portion of said endoscope insertion member in a distal direction away from said distal end portion of said endoscope insertion member.

21. The surgical kit defined in claim 18 wherein said coupling element is connected to a tube insertable through at least one of said tubular channel and said working channel.
